(19) United States
(12) Patent Application Publication
(10) Pub. No.: US 2023/0372707 A1
(43) Pub. Date: Nov. 23, 2023

(54) DYNAMIC ELECTROPORATION

(71) Applicant: Cochlear Limited, Macquarie University, NSW (AU)

(72) Inventor: Wolfram Frederik DUECK, Berlin (DE)

(21) Appl. No.: 18/044,496

(22) PCT Filed: Sep. 2, 2021
(86) PCT No.: PCT/IB2021/058028

§ 371 Date: Mar. 8, 2023

Related U.S. Application Data
(60) Provisional application No. 63/078,556, filed on Sep. 15, 2020.

(51) Int. Cl. A61N 1/32

(52) U.S. Cl. CPC A61N 1/327

(57) ABSTRACT

Presented herein are techniques for dynamic electroporation of the cells of a recipient of an implantable medical device. A carrier member with one or more electroporation electrodes is positioned in a spatial region proximate to cells of a recipient. The one or more electroporation electrodes are electrically connected to an external electroporation device and a treatment material to be transferred into the cells of the recipient is delivered proximate to the cells. The carrier member is moved through the spatial region while a series of electroporation signals, generated by the external electroporation device, are applied to the electroporation electrodes to generate an electroporation electrical field proximate to the cells. Due to the movement of the carrier member (and thus the electroporation electrodes) a location of a locus of the electroporation electrical field changes, over time, within the spatial region.

### BACKGROUND

### Field of the Invention

The present invention relates generally to dynamic electroporation of cells within a recipient of an implantable medical device.

### Related Art

Medical devices have provided a wide range of therapeutic benefits to recipients over recent decades. Medical devices can include internal or implantable components/devices, external or wearable components/devices, or combinations thereof (e.g., a device having an external component communicating with an implantable component). Medical devices, such as traditional hearing aids, partially or fully-implantable hearing prostheses (e.g., bone conduction devices, mechanical stimulators, cochlear implants, etc.), pacemakers, defibrillators, functional electrical stimulation devices, and other medical devices, have been successful in performing lifesaving and/or lifestyle enhancement functions and/or recipient monitoring for a number of years.

The types of medical devices and the ranges of functions performed thereby have increased over the years. For example, many medical devices, sometimes referred to as “implantable medical devices,” now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components.

### SUMMARY

In one aspect, a method is provided. The method comprises: inserting a carrier member comprising a plurality of electroporation electrodes into a spatial region within the body of a recipient; introducing a treatment material into the spatial region; and dynamically electroporating a population of cells located adjacent to the spatial region to transfer the treatment material into the population of cells.

In another aspect, a method is provided. The method comprises: positioning one or more electroporation electrodes within a spatial region adjacent to cells in the body of a recipient, wherein the one or more electroporation electrodes are electrically connected to an external electroporation device; delivering a treatment material configured for transfer into the cells of the recipient into the spatial region; and applying a series of current pulses from the external electroporation device to the one or more electroporation electrodes to generate at least one dynamic electroporation electrical field proximate to the cells while simultaneously moving the one or more electroporation electrodes along a path through the spatial region.

In another aspect, a method is provided. The method comprises: inserting a carrier member comprising a plurality of electroporation electrodes into a spatial region within the body of a recipient; introducing a treatment material into the spatial region; moving the carrier member through the spatial region; and while the carrier member is moved through the spatial region, delivering a series of electroporation signals to the electroporation electrodes to generate a dynamic electroporation electrical field where a location of a locus of the dynamic electroporation electrical field changes, over time, within the spatial region.

### BRIEF DESCRIPTION OF THE DRAWINGS

Embodiments of the present invention are described herein in conjunction with the accompanying drawings, in which:

FIG. 1A is a schematic diagram illustrating a cochlear implant system comprising an electroporation sub-system, in accordance with certain embodiments presented herein;

FIG. 1B is another schematic diagram illustrating the cochlear implant system of FIG. 1A, in accordance with certain embodiments presented herein;

FIG. 2A is another schematic diagram illustrating a stand-alone electroporation sub-system, in accordance with certain embodiments presented herein;

FIG. 2B is a schematic diagram illustrating use of the stand-alone electroporation sub-system of FIG. 2A for dynamic electroporation, in accordance with certain embodiments presented herein;

FIG. 2C is a schematic diagram illustrating another use of the stand-alone electroporation sub-system of FIG. 2A for dynamic electroporation, in accordance with certain embodiments presented herein;

FIGS. 3A, 3B, and 3C are a series of schematic diagrams illustration dynamic electroporation, in accordance with certain embodiments presented herein; and

FIG. 4 is a flowchart of a method, in accordance with certain embodiments presented herein;

FIG. 5 is a flowchart of another method, in accordance with certain embodiments presented herein;

FIG. 6 is a schematic diagram illustrating another cochlear implant system, in accordance with certain embodiments presented herein;

FIG. 7A is a schematic diagram illustrating a portion of a stimulating assembly in a first arrangement, in accordance with certain embodiments presented herein; and

FIG. 7B is a schematic diagram illustrating the portion of the stimulating assembly of FIG. 7A in a second arrangement, in accordance with certain embodiments presented herein.

FIG. 7C is a schematic diagram illustrating a portion of a stimulating assembly, in accordance with certain embodiments presented herein.

### DETAILED DESCRIPTION

Electroporation refers to the application of an electrical field to a cell in a manner that creates an electrical potential (i.e., voltage difference) across the cell membrane that, in turn, temporarily opens up pores in the lipid bilayer membrane of the cell. The electrically opened pores may be used to, for example, allow a treatment material to enter the interior of the cell through the cell membrane (i.e., as the potential difference is applied to the cell, the electrically opened pores in the cell membrane allow material to flow into the cell). After the electrical potential is removed, the pores in the cell membrane close such that the treatment material remains in the cell. As such, electroporation may be useful with medical implants by altering the biological composition of the cells in a manner that enhances, enables, etc. operation of the medical implants.

For successful electroporation, a cell is typically exposed to an electrical field for a sufficient amount of time that enables a desired treatment material to migrate through the cell membrane. Such an electric field, sometimes referred to herein as an “electroporation electrical field,” has a local field strength in the range of approximately 50 micro Volts per micro meter (uV/um) to approximately 500 uV/um (In general, the voltage needed to create a field having a strength in this range that is sufficient for electroporation heavily depends on the geometry, relative position, and distance of the anode and cathode electrode, and also on the medium between electrodes and cells.

There are several types of implantable medical devices that operate by delivering electrical (current) stimulation to the nerves, muscle, tissue fibers, or other cells of a recipient. These implantable medical devices typically deliver current stimulation to compensate for a deficiency in the recipient. For example, certain implantable medical device, such as cochlear implants, are often proposed when a recipient experiences sensorineural hearing loss due to the absence or destruction of the cochlear hair cells, which transduce acoustic signals into nerve impulses. Auditory brainstem stimulators are another type of implantable medical device prostheses that might be proposed when a recipient experiences sensorineural hearing loss due to damage to the auditory nerve.

Cochlear implants have been used to perform “static” electroporation (i.e., where the electrode(s) delivering/generating the electroporation electrical field do not move during the electroporation. For example, one or more electrodes are positioned in the inner ear (cochlea), and a solution containing a treatment material to be transferred (e.g., a viral vector comprising foreign genetic material) is introduced into the inner ear. Once in position, current is delivered to cells adjacent to at least one of the one or more inserted electrodes to provide an electrical shock to those cells adjacent to the at least one electrode. That is, a high voltage electric field is delivered to the cells that causes pores within the membrane of those cells to open, thereby allowing the treatment material to enter the cell (e.g., the cell membrane either temporarily (cell transfer) or permanently (cell ablation) become permeable allowing substances which normally cannot enter into a target cell to cross the cell membrane into the interior of the target cell).

Static electroporation has been used for a variety of in-vitro and in-vivo applications including scientific research, cancer treatment, gene therapy and for vaccination. The efficiency of static electroporation is determined by how many cells are successfully electroporated. Typical approaches to increasing yield include increasing the concentration of the material to be transferred into the cells relative to the number of cells, varying electroporation voltages or field strength, or optimizing a number of electrical pulses. However, with static electroporation, the electroporation is limited to nearby cells based on position and size of the electrodes, and therefore, efficacy of transformation is sub-optimal. Although a larger electric field may be generated in order to reach more cells, such an approach may lead to damage to cells within the electric field. Accordingly, static electroporation has several disadvantages and is not well suited for generating optimal transfer efficiencies.

Accordingly, presented herein are techniques that use “dynamic electroporation” to transfer a treatment material into cells of a recipient. In accordance with the dynamic electroporation techniques presented herein, a “dynamic” (i.e., moving) electroporation electrical field is generated via (by) one or more electroporation electrodes that are moved through a spatial region within, near, or adjacent to (collectively and generally referred to as “a spatial region proximate to”) target tissue (cells) within the body of a recipient. However, in accordance with the dynamic electroporation techniques presented herein, the locus (e.g., central point) of the dynamic electroporation electrical field, which is generated by delivery of a series of electroporation signals to the electroporation electrodes, changes over time. That is, the physical location of the locus of the dynamic electroporation electrical field is time-varying and, in general, progress through a spatial region in the body of a recipient.

In certain embodiments presented herein, the time-varying change in the location of the locus of the dynamic electroporation electrical field is a result of the movement of the electroporation electrodes within a spatial region of the recipient. That is, the change in the location of the locus of the dynamic electroporation electrical field corresponds to a change in the physical positioning of the electroporation electrodes used to generate the dynamic electroporation electrical field.

In general, dynamic electroporation results in electroporation of the cell population adjacent to the spatial region through which the electroporation electrodes that are moved (e.g., cells adjacent to the length of the path/track of the moving electrodes). That is, dynamic electroporation techniques may be used to transfer treatment material into cells along the path of the electrode and are able to target a greater number of cells as compared to traditional static electroporation techniques (e.g., techniques that use a stationary electrode array with multiple electrode pairs (e.g., anode and cathode) along with a complex set-up). As such, when compared to static electroporation, dynamic electroporation enables a larger population of cells to be electroporated without increasing the size of the electrodes, without increasing the strength of the electrical field, without using a larger number of electrodes, and potentially in a shorter period of time. In contrast to conventional static electroporation techniques, dynamic electroporation may electroporate an large area with a single pair of electrodes, which is a simpler set-up that is able to achieve the same or even higher number of total electroporated cells. This leads to improved yields and less damage to cells.

As used herein, the dynamic electroporation is delivered via one or more electroporation electrode contacts (electroporation electrodes) that are disposed in (e.g., positioned in/on) a carrier member. The electroporation electrodes are electrically connected to an external current source (e.g., a current source located outside of the body of the recipient), sometimes referred to herein as an “electroporation device,” that generates the electroporation current signals. The electroporation electrodes and the external current source are sometimes collectively referred to as a dynamic electroporation sub-system. As described further below, dynamic electroporation can be delivered via a stand-alone dynamic electroporation sub-system (e.g., a dynamic electroporation sub-system that is separate from an implantable medical device) or via a dynamic electroporation sub-system integrated with an implantable medical device.

Dynamic electroporation electrodes in accordance with embodiments presented herein are configured/arranged such that the external electroporation device delivers periodic pulses of current to the electroporation electrodes as the carrier member is moved through the spatial region proximate to the target tissue within the body of a recipient. In certain embodiments, the electroporation is performed as the electroporation electrodes are moved from a proximal location (e.g., a location relatively closer to the outside of the recipient's body) towards a distal location (e.g., a location relatively further away from the outside of the recipient's body) or, stated differently, as the electroporation electrodes are moved from an insertion point/position towards a final/destination point/position. In certain embodiments, the electroporation is performed as the electroporation electrodes are moved from a distal location towards a proximal location or, stated differently, as the electroporation electrodes are moved from a final/destination point/position towards an insertion point/position. In still other embodiments, the electroporation is performed as the electroporation electrodes are moved from a proximal location towards a distal location and as the electroporation electrodes are moved from the distal location towards the proximal location. That is, the dynamic electroporation can be performed while the carrier member is inserted (i.e., as the carrier member is moved from the insertion point to the destination point) and/or while the carrier member is removed/retracted (i.e., as the carrier member is moved from the destination point to the insertion point).

As noted above, a dynamic electroporation electrical field is used to open pores in the cell membrane, which enable a treatment material to enter the cells. That is, by delivering periodic pulses of current to the electroporation electrodes as the electroporation electrodes are moved through the spatial region, a dynamic (moving) electroporation electrical field is generated where the location of the locus of the electrical field changes, over time. Due to the change in the location of the locus of the dynamic electroporation electrical field, over-time, cells along the insertion path (adjacent to the spatial region) are subject to an electric field strength high enough to generate pores in the cell membranes, in order to maximize the number of cells that uptake the treatment material. As such, the treatment material is introduced into the proximity of the cells to be electroporated before (prior to) delivery of the dynamic electroporation electrical field. In certain embodiments, as the electroporation carrier member is moved, a solution containing the treatment material to be transferred into the cells is pumped into the spatial region proximate to the target tissue (e.g., into the cochlea).

As noted above, the electroporation sub-systems provided herein may, in certain embodiments, operate as a standalone device (e.g., not part of an implantable medical device). However, in other embodiments, the electroporation sub-systems provided herein may be part of (e.g., partially or fully integrated in) an implantable medical device. There are several types of implantable medical devices that deliver stimulation signals (current signals) to compensate for a deficiency in a recipient. Merely for ease of illustration, the embodiments presented herein are primarily described with reference to one type of implantable medical device, namely a cochlear implant system. However, it is to be appreciated that the techniques presented herein may also be used with a variety of other implantable medical devices and/or systems. For example, the techniques presented herein may be used with other auditory prosthesis systems, including middle ear auditory prosthesis systems (middle ear implant systems), bone conduction device systems, direct acoustic stimulator systems, electro-acoustic prosthesis systems, auditory brain stimulator systems, etc. The techniques presented herein may also be used with systems that comprise or include tinnitus therapy devices, vestibular devices (e.g., vestibular implants), visual devices (i.e., bionic eyes), sensors, pacemakers, drug delivery systems, including systems/devices to deliver DNA vaccinations and/or other compounds (e.g., as cis-platin for electrochemotherapy, etc.), defibrillators, functional electrical stimulation devices, catheters, seizure devices (e.g., devices for monitoring and/or treating epileptic events), sleep apnea devices, electroporation devices, etc.

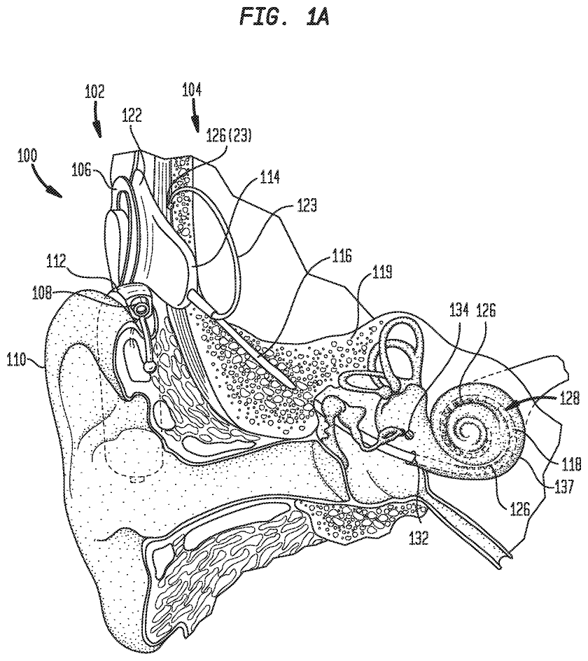

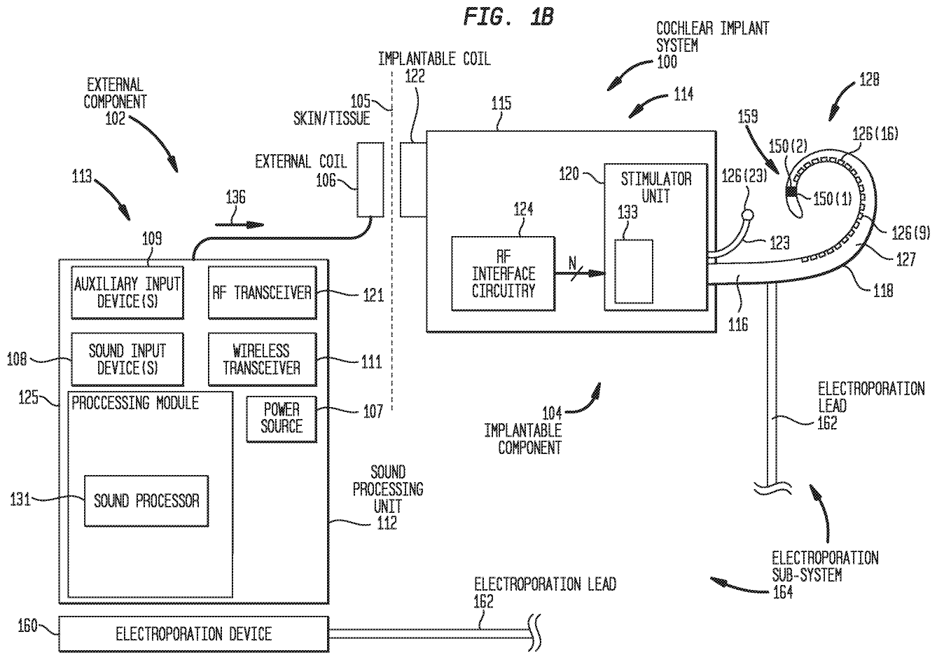

FIG. 1A is a schematic diagram of an exemplary cochlear implant system 100 configured to implement aspects of the techniques presented herein, namely configured for use in performing dynamic electroporation. FIG. 1B is a block diagram of the cochlear implant system 100. For ease of illustration, FIGS. 1A and 1B will be described together.

The cochlear implant system 100 comprises an external component 102 and an internal/implantable component 104, sometimes referred to herein as “cochlear implant 104.” The external component 102 is directly or indirectly attached to the body of the recipient and typically comprises an external coil 106 and, generally, a magnet (not shown in FIG. 1A) fixed relative to the external coil 106. The external component 102 also comprises one or more input elements/devices 113 for receiving input signals at a sound processing unit 112. In this example, the one or more one or more input devices 113 include sound input devices 108 (e.g., microphones positioned by auricle 110 of the recipient, telecoils, etc.) configured to capture/receive input signals, one or more auxiliary input devices 109 (e.g., audio ports, such as a Direct Audio Input (DAI), data ports, such as a Universal Serial Bus (USB) port, cable port, etc.), and a wireless transmitter/receiver (transceiver) 111, each located in, on, or near the sound processing unit 112.

The sound processing unit 112 also includes, for example, at least one battery 107, a radio-frequency (RF) transceiver 121, and a processing module 125. The processing module 125 may comprise a number of elements, including a sound processor 131.

In the examples of FIGS. 1A and 1B, the sound processing unit 112 is a behind-the-ear (BTE) sound processing unit configured to be attached to, and worn adjacent to, the recipient's ear. However, it is to be appreciated that embodiments of the present invention may be implemented by sound processing units having other arrangements, such as an off-the-ear (OTE) sound processing unit (i.e., a component having a generally cylindrical shape and which is configured to be magnetically coupled to the recipient's head), etc., a mini or micro-BTE unit, an in-the-canal unit that is configured to be located in the recipient's ear canal, a body-worn sound processing unit, etc.

Returning to the example embodiment of FIGS. 1A and 1B, the implantable component (cochlear implant) 104 comprises an implant body (main module) 114, a lead region 116, and an intra-cochlear stimulating assembly 118, all configured to be implanted under the skin/tissue (tissue) 105 of the recipient. The implant body 114 generally comprises a hermetically-sealed housing 115 in which RF interface circuitry 124 and a stimulator unit 120 are disposed. As described further below, the stimulator unit 120 comprises stimulation electronics 133. The stimulation electronics 133 comprises, among other elements, one or more current sources on an integrated circuit (IC). The implant body 114 also includes an internal/implantable coil 122 that is generally external to the housing 115, but which is connected to the RF interface circuitry 124 via a hermetic feedthrough (not shown in FIG. 1B).

As noted, stimulating assembly 118 is configured to be at least partially implanted in the recipient's cochlea 137. Stimulating assembly 118 includes a carrier member 127 and a plurality of longitudinally spaced intra-cochlear electrodes 126 disposed in/on the carrier member 127. The intra-cochlear electrodes 126 collectively form a contact or electrode array 128 configured to deliver electrical stimulation signals (current signals) to the recipient's cochlea and/or to sink stimulation signals from the recipient's cochlea. FIG. 1A illustrates a specific arrangement in which stimulating assembly 118 comprises twenty-two (22) intra-cochlear electrodes 126, labeled as electrodes 126(1) through 126(22). It is to be appreciated that embodiments presented herein may be implemented in alternative arrangements having different numbers of intra-cochlear electrodes.

Stimulating assembly 118 extends through an opening in the recipient's cochlea (e.g., cochleostomy, the round window, etc.) and has a proximal end connected to stimulator unit 120 via lead region 116 and a hermetic feedthrough (not shown in FIG. 1B). Lead region 116 includes a plurality of conductors (wires) that electrically connect the electrodes 126 to the stimulator unit 120.

Also shown in FIG. 1A is an extra-cochlear electrode 126(23). The extra-cochlear electrode 126(23) is an electrode contact that is configured to, for example, deliver electrical stimulation to the recipient's cochlea and/or to sink current from the recipient's cochlea. The extra-cochlear electrode 126(23) is connected to a reference lead 123 that includes one or more conductors that electrically couple the extra-cochlear electrode 126(23) to the stimulator unit 120.

As described further below, the intra-cochlear electrodes 126(1)-126(22) and the extra-cochlear electrode 126(23) can be used post-operatively to stimulate the cochlea 137 of the recipient (i.e., operate as delivery or return paths for current signals to the cochlea 137) that evoke a hearing perception. As such, for ease of description, the intra-cochlear electrodes 126(1)-126(22) and the extra-cochlear electrode 126(23) are sometimes referred to herein as “stimulation electrodes.”

FIGS. 1A and 1B also illustrate that the stimulating assembly 118 includes two (2) electrode contacts (electrodes) 150(1) and 150(2). As described further below, the electrodes 150(1) and 150(2) are electrically connected to an external electroporation device 160 (shown in FIG. 1B) via an electrporation lead 162 (e.g., one or more wires). As described elsewhere herein, the electrodes 150(1) and 150(2) can be used to electroporate the recipient's cochlea nerve cells during implantation of the stimulating assembly 118 into the cochlea 137. That is, the electrodes 150(1) and 150(2) may be configured to source, sink, or both source and sink electroporation signals (generated by external electroporation device 160) that result in the application of a dynamic electroporation electrical field to the nerve cells of the cochlea 137. Thereafter, the electrodes 150(1) and 150(2) can be electrically isolated from the external electroporation device 160 (e.g., electrically disconnected). Although shown as two electrodes located at the distal end 159 of the stimulating assembly 118, the more than two electrode electrodes may be present and used for electroporation.

As noted elsewhere herein, electrodes configured for use in performing electroporation, such as electrodes 150(1) and 150(2), are referred to as “electroporation electrodes.” The electroporation electrodes 150(1)/150(2), and the external electroporation device 160, are collectively referred to herein as a “dynamic electroporation sub-system” 164. In the embodiments of FIGS. 1A and 1B, the dynamic electroporation system 164 is referred to as being integrated with the cochlear implant system 100.

In the embodiments of FIGS. 1A and 1B, the electroporation electrodes 150(1) and 150(2) are structurally distinguishable from the intra-cochlear electrodes 126(1)-126(22) in that the electroporation electrodes are not connected to the stimulator unit 120 (whereas the intra-cochlear electrodes 126(1)-126(22 must be connected to the stimulator unit 120 to enable operation thereof). In particular, the high voltages associated with electroporation could damage the stimulator unit 120 and, as such, the electroporation electrodes 150(1) and 150(2) are electrically isolated from the stimulator unit 120. It also follows that, due to the electrical connection between the intra-cochlear electrodes 126(1)-126(22) and the stimulator unit 120, the intra-cochlear electrodes 126(1)-126(22) cannot be used to perform electroporation.

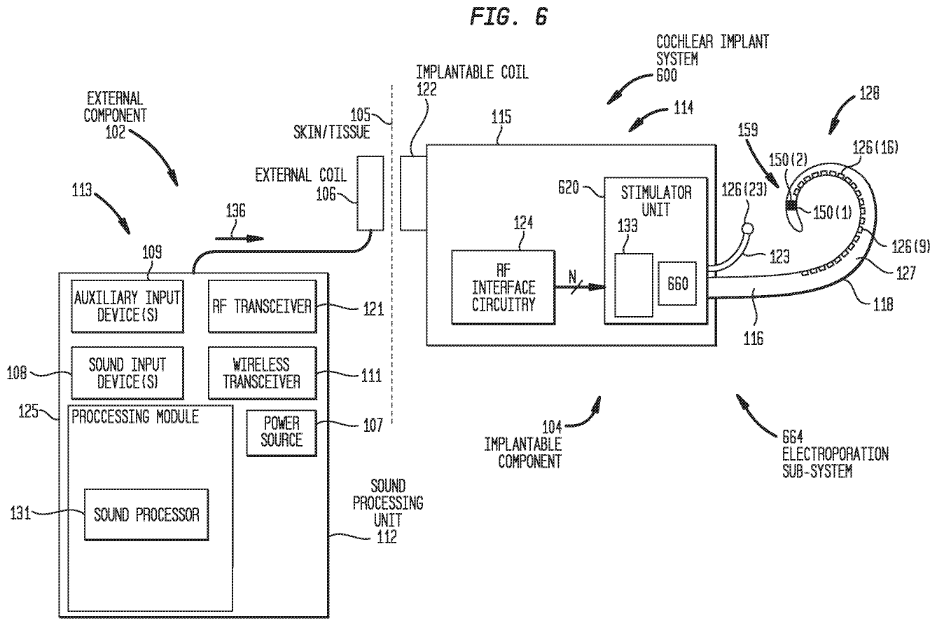

As noted, in the example of FIGS. 1A and 1B, the electroporation electrodes 150(1) and 150(2) are electrically connected to an external electroporation device 160. However, in certain alternative embodiments, the stimulator unit 120 may be designed to generate and handle the larger electroporation voltages. As such, in such embodiments, the electroporation electrodes 150(1) and 150(2) could be connected to the stimulator unit 120, which in turn includes an electroporation signal generator, for generation and delivery of the electroporation signals (e.g., no connection to an external source is needed to generate the electroporation signals). Such an arrangement is shown in FIG. 6.

More specifically, FIG. 6 illustrates a cochlear implant system 600 that is substantially similar to cochlear implant system 100 of FIGS. 1A-1D. However, in this example, the implantable component includes a stimulator 620 that be designed to generate and handle the larger electroporation voltages. To this end, the where the stimulator unit 620 includes an on-board electroporation signal generator 660. The electroporation electrodes 150(1)/150(2), and the on-board electroporation signal generator 660, are collectively referred to herein as a “dynamic electroporation sub-system” 664. In the embodiment of FIG. 6, the dynamic electroporation system 664 is referred to as being integrated with the cochlear implant system 600.

Electroporation may have a number of associated purposes. In certain examples, the electroporation is used to open the pores in the cells in the presence of treatment materials including/comprising a therapeutic agent in order to enable an effective amount of the therapeutic agent to enter the electroporated (opened) cells. As used herein, the term “therapeutic agent” may include, but is not limited to, biological or bioactive substances, chemicals, pharmaceutical agents, nanoparticles, ions, including nucleic acids (e.g., Deoxyribonucleic acid (DNA), DNA cassettes, cDNA, or plasmids, Ribonucleic acid (RNA) molecules, RNAi, etc.), proteins, peptides (e.g., Brain-derived neurotrophic factors, etc.), hormones, etc. Therefore, in accordance with certain embodiments, prior to electroporation, a therapeutic agent may first be delivered to the cochlea 137. Such a therapeutic agent may be delivered in a number of different manners, such as through an implantation tool, substance delivery device (e.g., lumen, syringe, etc.), a lumen within the carrier member 127, a coating on the carrier member 127, etc.

As noted, the cochlear implant system 100 includes the external coil 106 and the implantable coil 122. The coils 106 and 122 are typically wire antenna coils each comprised of multiple turns of electrically insulated single-strand or multi-strand platinum or gold wire. Generally, a magnet is fixed relative to each of the external coil 106 and the implantable coil 122. The magnets fixed relative to the external coil 106 and the implantable coil 122 facilitate the operational alignment of the external coil with the implantable coil. This operational alignment of the coils 106 and 122 enables the external component 102 to transmit data, as well as possibly power, to the implantable component 104 via a closely-coupled wireless link formed between the external coil 106 with the implantable coil 122. In certain examples, the closely-coupled wireless link is a radio frequency (RF) link. However, various other types of energy transfer, such as infrared (IR), electromagnetic, capacitive and inductive transfer, may be used to transfer the power and/or data from an external component to an implantable component and, as such, FIG. 1B illustrates only one example arrangement.

As noted above, sound processing unit 112 includes the processing module 125. The processing module 125 is configured to convert input audio signals into stimulation control signals 136 for use in stimulating a first ear of a recipient (i.e., the processing module 125 is configured to perform sound processing on input audio signals received at the sound processing unit 112). Stated differently, the sound processor 131 (e.g., one or more processing elements implementing firmware, software, etc.) is configured to convert the captured input audio signals into stimulation control signals 136 that represent stimulation signals for delivery to the recipient. The input audio signals that are processed and converted into stimulation control signals may be audio signals received via the sound input devices 108, signals received via the auxiliary input devices 109, and/or signals received via the wireless transceiver 111.

In the embodiment of FIG. 1B, the stimulation control signals 136 are provided to the RF transceiver 121, which transcutaneously transfers the stimulation control signals 136 (e.g., in an encoded manner) to the implantable component 104 via external coil 106 and implantable coil 122. That is, the stimulation control signals 136 are received at the RF interface circuitry 124 via implantable coil 122 and provided to the stimulator unit 120. The stimulator unit 120 is configured to utilize the stimulation control signals 136 to generate stimulation signals (e.g., current signals) for delivery to the recipient's cochlea via one or more stimulation electrodes 126(1)-126(22). In this way, cochlear implant system 100 electrically stimulates the recipient's auditory nerve cells, bypassing absent or defective hair cells that normally transduce acoustic vibrations into neural activity, in a manner that causes the recipient to perceive one or more components of the input audio signals.

Due to the confining bony structure of the cochlea, insertion of a stimulation electrode array is comparatively easy, safe and provides a stable body-implant interface, all of which have contributed to the success of the cochlear implant. In addition, advanced mechanical designs has given small improvements in performance. However, one of the key performance-limiting factors in conventional cochlear implants is the distance between the stimulating intra-cochlear electrodes and the corresponding stimulated spiral ganglion cells. If the distance between electrodes and the stimulated spiral ganglion cells could be reduced, it may be possible to, for example: create additional stimulation sites that, in turn, may deliver better frequency discrimination to the recipient, enable use of lower stimulation currents and, accordingly, reduce implant power consumption, and/or enable use of lower stimulation voltages, and, accordingly reduce implant size and power dissipation. It has been proposed to inject neural growth factors into the cochlea during surgery to entice the nerves to grow towards the stimulation electrodes. However, the injected neural growth factors generally dissipate before any significant benefit is obtained. One possible solution to this key problem is to insert neural growth factor genes into cells in the cochlear via cell electroporation during surgical implantation of an intra-cochlear stimulating assembly.

As noted, electroporation refers to the application of an electrical field to a cell such that pores are opened in the cell membrane. When these cells are opened in the presence of a therapeutic agents, such as neural growth factor genes, the therapeutic agents may enter the cell through the cell membrane. After the electrical potential is removed, the pores in the cell membrane close such that the therapeutic agents remain in the cell.

Also as noted, successful electroporation requires a cell to be exposed to an electrical field utilizing a voltage that is sufficient to create pores in a membrane. Presented herein are techniques that enable electroporation of cells (e.g., the cochlea nerve) along a pathway for cochlear implantation through application of a dynamic electroporation electrical field.

More specifically, referring to the arrangement of FIGS. 1A and 1B, the electroporation electrodes 150(1) and 150(2) are electrically connected to the external electroporation device 160 via an electroporation lead 162. The electroporation electrodes 150(1) and 150(2) are used to dynamically electroporate the recipient's cochlea nerve cells during implantation of the stimulating assembly 118 into the cochlea 137 (i.e., while the electrode assembly 118 is inserted into the cochlea 137). In certain examples, positioning the electroporation electrodes 150(1) and 150(2) at a distal end 159 of the carrier member, a lower voltage may be supplied to the electroporation electrode along the path, allowing a sufficient number of cells to be transferred.

FIGS. 1A and 1B illustrate an arrangement in which the cochlear implant system 100 includes an external component. As noted elsewhere herein, it is to be appreciated that embodiments of the present invention may be implemented in cochlear implant systems having alternative arrangements and/or in other types of implantable medical devices. As noted above, merely for ease of description, the techniques presented herein have primarily been described herein with reference to a cochlear implant system. However, it is to be appreciated that the techniques presented herein may also be used with a variety of other implantable or non-implantable medical devices and related medical device systems. For example, the techniques presented herein may be used with other auditory prosthesis systems, including middle ear auditory prosthesis systems (middle ear implant systems), bone conduction device systems, direct acoustic stimulator systems, electro-acoustic prosthesis systems, auditory brain stimulator systems, etc. The techniques presented herein may also be used with systems that comprise or include tinnitus therapy devices, vestibular devices (e.g., vestibular implants), visual devices (i.e., bionic eyes), sensors, pacemakers, drug delivery systems, including devices for delivery of vaccinations, chemotherapy, etc. (e.g., where there is no device left behind in the body), defibrillators, functional electrical stimulation devices, catheters, seizure devices (e.g., devices for monitoring and/or treating epileptic events), sleep apnea devices, electroporation devices, etc. However, merely for ease of description, further details of the techniques presented herein will generally be described with reference to cochlear implants systems

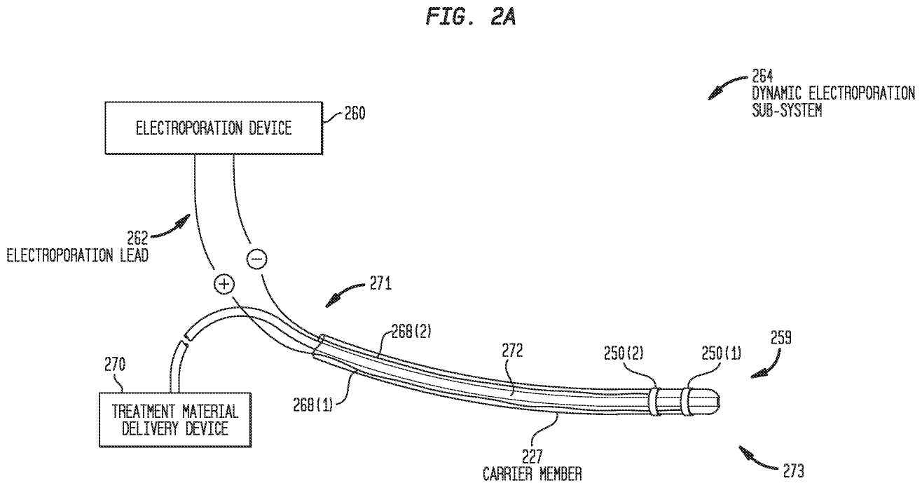

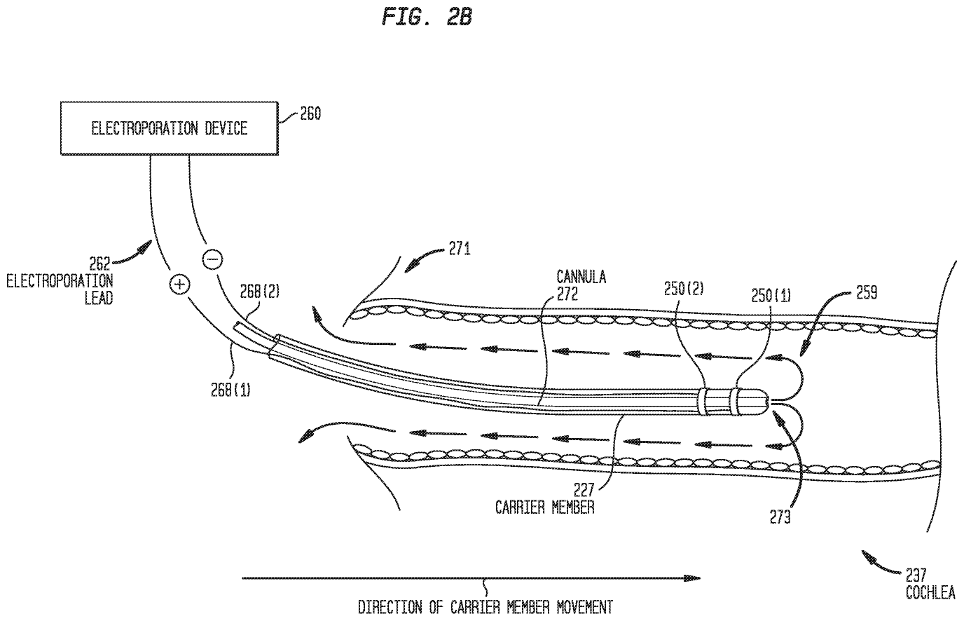

As noted above, certain embodiments presented herein may use electrodes that are dedicated for use during electroporation only (i.e., electroporation electrodes), as well as intra-cochlear electrodes and one or more extra-cochlear electrodes (collectively stimulation electrodes that may be used post-operatively for stimulating the cochlea of a recipient). Also as noted above, the electroporation electrodes may be integrated into the same carrier member (e.g., silicone or elastomer body) as the intra-cochlear electrodes. Incorporating electroporation electrodes into the same carrier member as the intra-cochlear electrodes may make the geometry (and hence the electrical field which governs the electroporation process) well-defined during electroporation. However, in other embodiments the electroporation electrodes may be physically separate from the carrier member in which the intra-cochlear electrodes are disposed (e.g., part of an insertion tool, separate electrodes, etc.), referred to above as a “stand-alone dynamic electroporation sub-system.” FIGS. 2A, 2B, and 2C illustrate embodiments of one such stand-alone dynamic electroporation sub-system, in accordance with embodiments presented.

More specifically, FIG. 2A is a schematic diagram of a stand-alone dynamic electroporation sub-system 264, in accordance with certain embodiments presented herein, while FIGS. 2B and 2C are diagrams illustrating the stand-alone electroporation sub-system 264 partially inserted into the cochlea 237 of a recipient. For ease of description, FIGS. 2A-2C will be described together.

As shown, the stand-alone dynamic electroporation sub-system 264 comprises a carrier member (carrier member 227) in/on which two electroporation electrode contacts (electrodes) 250(1) and 250(2) are positioned/disposed. In the specific example of FIGS. 2A-2C, the electroporation electrodes 250(1) and 250(2) are positioned at a distal end (tip) 259 of the carrier member 227. The electroporation electrodes 250(1) and 250(2) are electrically connected to an external electroporation device 260, which is configured to generate electroporation signals (current signals) to be delivered via the electroporation electrodes 250(1) and 250(2). More specifically, in this example, carrier member 227 includes two isolated electrical conductors (e.g. wires) 268(1) and 268(2), each of which terminate at one of the electroporation electrodes 250(1) and 250(2), respectively. The electrical conductors 268(1) and 268(2) extend through the electroporation lead 262 to the electroporation device 260.

As noted above, in order to perform dynamic electroporation, a treatment is introduced into the proximity of the cells to be electroporated before (prior to) delivery of the dynamic electroporation electrical field. As such, in the embodiments of FIGS. 2A-2C, the dynamic electroporation sub-system 264 comprises a treatment material delivery device 270. In these embodiments, as the carrier member 227 is moved through the cochlea, a solution containing the treatment material to be transferred into the cochlea cells is pumped into the spatial region proximate to the target tissue (e.g., into the cochlea 237). More specifically, the treatment material delivery device 270 is fluidically coupled to a proximal end 271 of an elongate cannula 272 extending longitudinally through the carrier member 227. The cannula 272 has a distal opening 273 located at the distal end 259 of the carrier member 227 (e.g., for supplying treatment material to be transferred into cells).

Treatment material delivery device 270 may be electrically operated (e.g., by a pump, pressurized reservoir, etc.) or may be manually operated (e.g., a syringe, manual pump, etc.) to supply a solution comprising the treatment material to be transferred into cells. The flow rate of the fluid delivery device may be set at a constant or variable flow rate to ensure an effective amount of the therapeutic agent is delivered to the appropriate location for electroporation.

As noted above, in accordance with the dynamic electroporation techniques presented herein, a dynamic electroporation electrical field is generated via (by) the electroporation electrodes 250(1) and 250(2) as they are moved through a spatial region proximate to target tissue (cells) within the body of a recipient. In the example of FIGS. 2A-2C, the spatial region proximate to target is the cochlea 227. During dynamic electroporation, the electroporation signals may be delivered repeatedly, periodically, etc., via the electroporation electrodes 250(1) and 250(2) as the carrier member 227 is inserted, retracted, or inserted and retracted into the cochlea. As a result, electroporation will occur along the entire length of the cochlea (at different points in time during the insertion). In the example of FIG. 2B, the dynamic electroporation is performed while the carrier member 227 is inserted into the cochlea 237 (as the carrier member is moved from the insertion point to the destination point, while in the example of FIG. 2C the dynamic electroporation is performed while the carrier member 227 is removed/retracted into the cochlea 237 (i.e., as the carrier member is moved from the destination point to the insertion point).

As noted above, in the embodiments of FIGS. 2A-2C, two electroporation electrodes 250(1) and 250(2) are positioned at the distal end (tip) 259 of the carrier member 227. Also as noted above, it is to be appreciated that the use of two electroporation electrodes is merely illustrative and that, in other embodiments, four, six, eight, ten, or more electrodes may positioned in/on the carrier member 227. In certain embodiments, the electroporation electrodes are arranged at, or begin at, proximate to the distal end 259 to enable electroporation of cells along the path of movement. However, in other embodiments, the electroporation electrodes can be arranged at other areas of the carrier member 227.

In certain, a number of electroporation electrodes can be arranged as multiple different anode/cathode pairs. The use of multiple different anode/cathode pairs during dynamic electroporation can enable the delivery of different electroporation pulses at each pair. It might be beneficial to apply pulses with difference attributes, such as with increasing voltage or amplitude, changing polarity, etc. so that when the array is moved the cells are subjected to a loci of increasing (or decreasing) field strength and changing direction. Such changes in field direction may be beneficial to the electroporation process to, for example, increase the amount of electroporated cells. Alternating the voltage amplitude and polarity of the electroporation pulses could also be implemented with a single electrode pair.

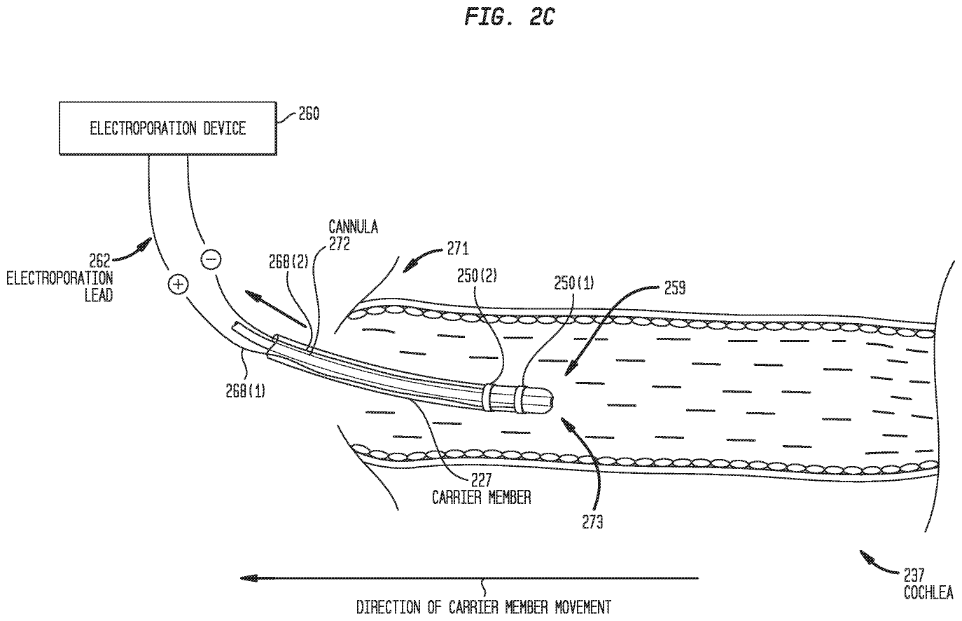

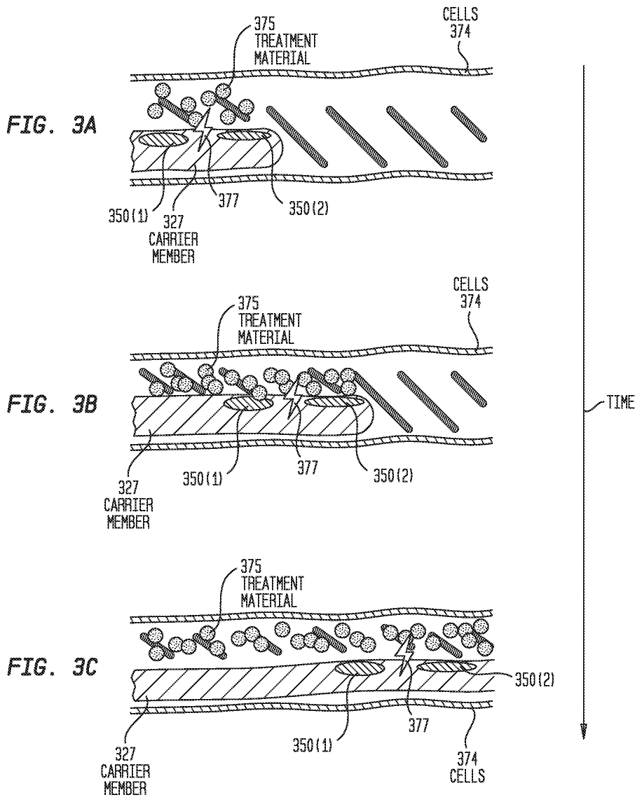

FIGS. 3A, 3B, and 3C are a series of schematic diagrams illustrating dynamic electroporation of cells 374 of a recipient, in accordance with embodiments presented herein. In the example 3A, 3B, and 3C, a carrier member 337 comprises two electroporation electrodes 350(1) and 350(2) and the dynamic electroporation is performed while the carrier member 337 is inserted (moved in a distal direction) through a spatial region proximate to the cells 374.

FIGS. 3A-3C illustrate that a treatment material 375 is introduced into the spatial region proximate to the cells 374 as the carrier member 327 is inserted (i.e., moved in a distal direction) into the spatial region. In particular, FIGS. 3A-3C show the progressive insertion of the electroporation electrode into the scala tympani or other spatial region adjacent to cells 374 of a recipient. FIGS. 3A-3C also schematically illustrate, at 377, the electroporation signals delivered to the cells 374 in order to enable transfer of the treatment material 375 into those cells.

In the embodiments of FIGS. 3A-3C, the electroporation signals 377 are delivered by a pair of electroporation electrodes 350(1) and 350(2) disposed at a distal end of the carrier member 327. As a result, the cells 374 adjacent to the path of movement of the carrier member 327 are electroporated along the length of the insertion path of the carrier member 327.

In general, the electroporation signals 377 comprise current waveforms of sufficient magnitude/amplitude and duration to cause pores to form in the cell membranes. Waveform shapes that may be used include, but are not limited to, square pulses, exponentially decaying pulses, step pulses, ramped pulses, alternating polarity, etc. The electroporation signals 377 may comprise, for example, monopolar pulses, biphasic pulses, etc. In addition, an electroporation signal 377 can be formed by any suitable number of pulses, including two, three, four, five, six, seven, eight, nine, ten, fifteen, twenty or more pulses. However, by minimizing the number of electroporation electrodes, damage to cells from high fields may also be minimized. With fewer electrodes, and by dynamically moving the electrode along a path, a smaller field may be generated and electroporation may still be performed with high efficiency. That is, in contrast to the use of one large field to perform electroporation (as is the case in conventional static arrangements), dynamic electroporation uses a series of smaller fields during travel/movement of the carrier member and, as a result, cells proximate to the electroporation electrodes may experience less damaging conditions during electroporation (when compared to damage occurring with the use of one large field to perform electroporation).

The attributes of the electroporation signals 377 may vary depending upon the cell type undergoing electroporation. For example, electroporation in the cochlea may necessitate higher voltages, different pulse rates, etc. than for other applications, such as neurostimulation of the spinal cord. In certain aspects, lower currents may be used for electroporation, as cells may be subjected to repeated pulses as the electroporation electrode is moved along a path adjacent to the cells.

In general, dynamic electroporation includes application of regular (e.g., continuous, periodic, etc.) current pulses to the cells 374 via the electroporation electrodes 350(1) and 350(2) as the carrier member 327 is moved along a path adjacent to the cells 374 to be electroporated. The cells 374 may form a tissue, organ or other structure in a recipient. In some aspects, movement of the carrier member 327 may include continuous movement or a series of discrete positions along the path (e.g., wherein the electroporation electrode tip moves along a path and pauses along a series of positions), wherein one or more pulses are applied at each position in a stepwise manner.

In the examples presented herein, such as shown in FIGS. 3A and 3B, two electroporation electrodes may be used to dynamically electroporate a cell population adjacent to the spatial region through which the electroporation electrodes are moved (e.g., cells adjacent to the length of the path/track of the moving electrodes). While embodiments are presented showing electroporation associated with directionality, these techniques may be used for any directionality, including insertion, removal/retraction, or a combination thereof. These techniques may be performed during multiple rounds of insertions and retractions.

As noted, the treatment material 375 is introduced into the spatial region, or at least in proximity to the cells to be electroporated, before the electroporation signals 377 are delivered to the cells 374. The treatment material 375 can be delivered in a number of different manners, as described above, and the treatment material 375 can have a number of different forms and/or attributes. In some aspects, the treatment material may comprise a therapeutic agent. Therapeutic agents may comprise biological or bioactive substances, chemicals, pharmaceutical agents, nanoparticles, ions, including nucleic acids (e.g., Deoxyribonucleic acid (DNA), DNA cassettes, cDNA, or plasmids, Ribonucleic acid (RNA) molecules, RNAi, etc.), proteins, peptides (e.g., Brain-derived neurotrophic factors, etc.), hormones, etc. or other agents that may be used to prevent or treat a disease or disorder. In some aspects, therapeutic agents that may be used to prevent or treat disorders or diseases of the inner ear may be transferred into cells using the techniques provided herein. Therapeutic agents may be suspended or dissolved in any suitable liquid, e.g., water, saline, etc. to form a solution. In general, the solution will have a level of conductivity suitable for performing electroporation.

A solution containing a therapeutic agent or other treatment material may be injected into the perilymph or other structure using any suitable device (e.g., needle, syringe, cannula, catheter, capillary tube, etc.) and may be injected simultaneously with, before, or after inserting or retracting the carrier member 327. For example, in certain embodiments, a treatment material could be delivered via a cannula inside the carrier member 327, delivered via a separate delivery device, released from the carrier member 327 (e.g., elution from silicone rubber, etc.), released from an excipient immobilized on the carrier member 327 such as from hydrogels (e.g., Poloxamer (Pluronic), hyaluronic acid (e.g., Healon), etc.), film forming systems (e.g., Unisun's PVA with nanoparticles, etc.), biodegradable matrices (e.g., PLGA, etc.), pellets (e.g., O-Ray, etc.), nanoparticles, etc.

In certain embodiments, the electroporation electrodes 350(1) and 350(2), or portions of the carrier member 327, are configured to form a seal with the walls of the spatial region (e.g., scala tympani) through which the carrier member 327 is moved. In such embodiments, the treatment material is introduced in this sealed volume so that the treatment material would not diluted be diluted by the recipient's bodily fluids.

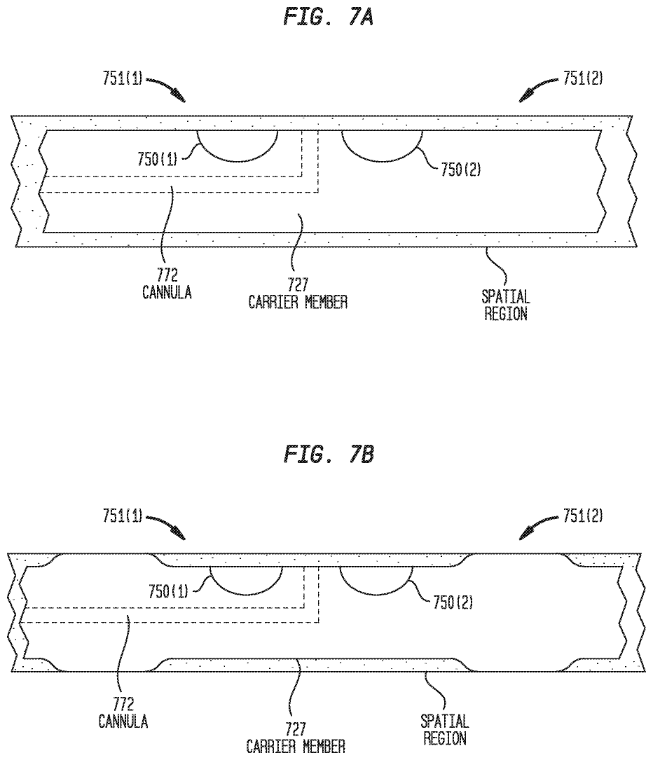

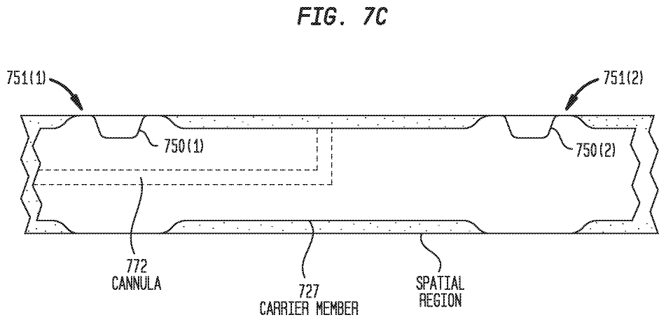

In certain embodiments, the electroporation electrodes 350(1) and 350(2), or portions of the carrier member 327, may expand to form a seal that brings the electroporation electrodes 350(1) and 350(2) in direct contact with the cells 374 Such direct contact could improve the electroporation efficacy. In such embodiments, contact pressure and material properties are selected so as not to cause mechanical damage to the cells during movement of the carrier member 327. In certain such embodiments, stepwise dynamic electroporation may be performed, wherein the carrier member 327 is positioned at a series of locations along the path. In this case, the carrier member 327 is moved to a first location and expands to form a seal wherein the electrode contacts are in direct contact with cells. The electroporation is performed at this first position and the carrier member 327 is subsequently contracted to remove the seal. The carrier member 327 is then moved to a second position and the above steps are repeated. This process can be performed iteratively at a number of different positions. Such an arrangement is shown in FIGS. 7A and 7B.

More specifically, FIGS. 7A and 7B illustrate a carrier member 727 that includes two electroporation electrodes 750(1) and 750(2) and a cannula 772. Disposed adjacent to the electroporation electrode 750(1) is an expandable portion 751(1), while disposed adjacent to the electroporation electrode 750(2) is an expandable portion 751(2). As shown in FIG. 7A, the expandable portions 751(1) and 751(2) each have a first (non-expanded) arrangement/configuration to facilitate insertion and movement of the carrier member 727. However, as shown in FIG. 7B, the expandable portions 751(1) and 751(2) each have a second (expanded) arrangement/configuration configured to form a seal with the walls of the spatial region (e.g., scala tympani) through which the carrier member 727 is moved. In the second arrangement of FIG. 7B, a treatment material can be introduced via the cannula 772 and the treatment material will be sealed in the volume bounded by the expandable portions 751(1) and 751(2), and which includes the electroporation electrodes 750(1) and 750(2). As shown in FIG. 7C, the electroporation electrode 750(1) can also or alternatively be located within the expandable portion 751(1), and the second electrode 750(2) located within the second expandable portion 751(2), to bring the electrodes in contact with the walls of the spatial region (e.g. cell lining on the inside wall of scala tympani).

In other aspects, space-filling and/or soft electrodes may be utilized to form a seal between the electroporation electrodes and the walls of the spatial region (e.g., scala tympani). This approach allows the electroporation electrodes to be positioned and a solution containing the treatment material to be delivered within the area formed by the seal. As a result, the treatment material will remain proximate to cells during electroporation. For example, conductive hydrogels (e.g., conductive hydrogels for medical applications, including composites of CPs (e.g., polypyrrole (PPy), polyaniline (PANI), polyethylene dioxythiophene (PEDOT)) and polysaccharides or proteins (e.g., alginates, gelatin/gelMA, collagen, chitosan, etc.) may be used to form the electroporation electrodes 350(1) and 350(2) and may expand to. The dehydrated, conductive hydrogel may be provided as a coating on the two metal electrode contacts. In contact with the perilymph, the hydrogel swells as it becomes more hydrated until contacting the cell covered spatial region. For example, co-electrochemical deposition of PEDOT and alginate hydrogel on an electrode contact site of a multichannel medical device (e.g., BT PEG-gelDOT coatings and BT PEDOT-PSS (BTPP) coatings, etc.), when hydrated, may expand to fill gaps between the electroporation electrode and the surface to which it is intended to contact. The above approach allows electroporation electrodes to be placed into close contact with target cells to be electroporated. Due to the material properties of the electroporation electrode, and low pressure applied to the cells, the electroporation electrode may be moved (e.g., inserted or retracted from the cochlea) without causing damage to the cells that it contacts.

In certain embodiments, electrophysiological feedback may be utilized to govern insertion and/or retraction speed of the carrier member 327. For example, electrophysiological measurements include but are not limited to electrical impedance measurements, neural response telemetry (NRT) measurements, Electrochochleography (EcochG) measurements, etc. For example, impedance may be used as a form of surgical feedback to measure, confirm and position the carrier member 327, as well as determine whether the carrier member 327 (or electrodes 350(1)/340(2)) is in contact with cells. In some aspects, electrophysiological feedback measurements may be performed with more than two electrodes.

NRT and/or EcochG, for example, may be used to assess tissue health and/or damage to neural structures during insertion of the carrier member 327. In aspects, EcochG may be used in combination with the electroporation processes described herein to prevent and/or minimize damage to the structures of the cochlea.

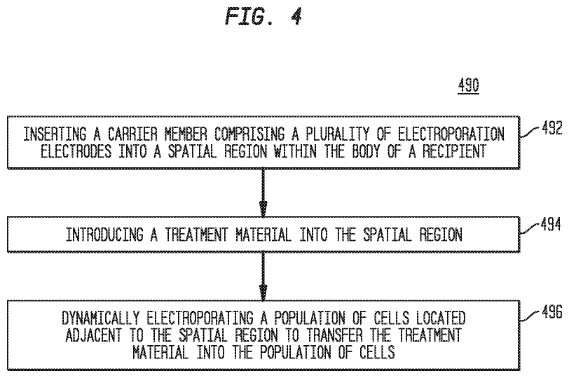

FIG. 4 is a flowchart of a method 490 in accordance with certain embodiments presented herein. Method 490 begins at 492 where a carrier member comprising a plurality of electroporation electrodes is inserted into a spatial region within the body of a recipient. At 494, a treatment material is introduced into the spatial region. At 496, a population of cells located adjacent to the spatial region are dynamically electroporated to transfer the treatment material into the population of cells.

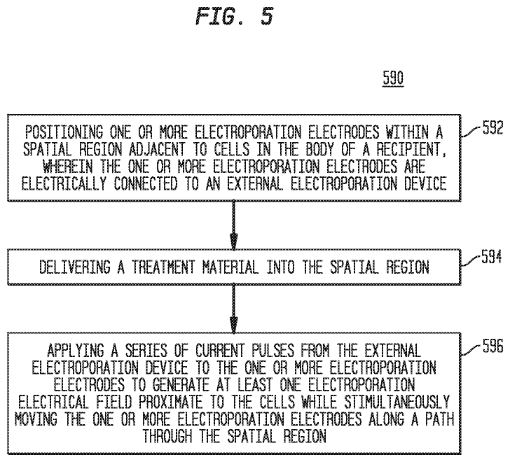

FIG. 5 is a flowchart of a method 590 in accordance with certain embodiments presented herein. Method 590 begins at 592 where one or more electroporation electrodes are positioned within a spatial region adjacent to cells in the body of a recipient. The one or more electroporation electrodes are electrically connected to an external electroporation device. At 594, a treatment material is delivered into the spatial region. At 596, a series of current pulses are applied from the external electroporation device to the one or more electroporation electrodes to generate at least one dynamic electroporation electrical field proximate to the cells while simultaneously moving the one or more electroporation electrodes along a path through the spatial region.

Advantages of present techniques include but are not limited to electroporation of a higher amount/number of cells, performing electroporation more evenly along a path, performing electroporation with less damage to the cells, and having a simpler setup that achieves the same or even a better result as a complex electrode array. Dynamic electroporation can also be beneficial when using penetrating needles to electroporate cells along any injection tract.

### EXAMPLES

An example protocol for dynamic electroporation to electroporate cells inside a scala tympani of the cochlea is provided. This protocol may be adapted to perform electroporation on other locations within the body and may be used with other designs that are suitable for electroporation in the other locations within the body.

Referring again to FIGS. 2A-C, the carrier member 227 is inserted into the scala tympani of the cochlea 337 through the round window membrane or cochleostomy, as shown in FIG. 2B. As noted, the two electrode contacts 250(1) and 250(2) are connected to the electroporation device 260 so as to receive and deliver current signals for generation of a dynamic electroporation electrical field and the cannula 272 is fluidically coupled to the treatment material delivery device 270.

Following the standard surgical approach, the cochlea 237 of the recipient is exposed and carrier member 227 is positioned at the insertion point into the scala tympani. After the carrier member 227 has reached its final position (deepest or most distal insertion point), the cannula 272 is used to inject a solution that contains the treatment material to be transferred into the recipient's target cells.

As the solution is introduced into the scala tympani, the volume of perilymph displaced by the influx of the solution, without buildup of significant positive pressure inside the scala tympani, is achieved by ensuring that the displaced perilymph is released at the insertion point (e.g., where the carrier member 227 enters the scala tympani). In one aspect, the opening of the scala tympani is sufficiently large so as not to form a seal against the electroporation electrode in order to allow the displaced perilymph to flow into the middle ear space. In this scenario, the injected solution containing material to be electroporated displaces the perilymph into the middle ear space, which results in bathing the cells of the scala tympani from the tip to base of the electroporation electrode in a solution comprising the material to be electroporated. Thus, perilymph is displaced by a material (e.g., a DNA solution) fed through the cannula at a selected rate during movement of the electrode along a path).

Subsequently, the electroporation device 260 is activated to deliver electroporation signals (current pulses), as appropriate, in order to electroporate the cells lining the inside wall of the scala tympani directly adjacent to the electroporation electrodes 250(1) and 250(2). While the electroporation signals are delivered, the carrier member 227 is slowly retracted (e.g., by a surgeon using tweezers, by a motorized retractor, etc.) until the electroporation electrode is removed completely from the scala tympani. While the electroporation electrode is being retracted, additional treatment material may be regularly or continually introduced into the scala tympani. As a result, cell electroporation may occur along the track of the two electroporation electrodes 250(1) and 250(2), where the track extends from the initial position of the tip 259 of the carrier member 327 to the insertion point of the scala tympani, shown as electroporated cells in FIG. 2C.

It is to be appreciated that the above described embodiments are not mutually exclusive and that the various embodiments can be combined in various manners and arrangements.

The invention described and claimed herein is not to be limited in scope by the specific preferred embodiments herein disclosed, since these embodiments are intended as illustrations, and not limitations, of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims.

1. A method, comprising:
inserting a carrier member comprising a plurality of electroporation electrodes into a spatial region within a body of a recipient;
introducing a treatment material into the spatial region; and
dynamically electroporating a population of cells located adjacent to the spatial region to transfer the treatment material into the population of cells.

2. The method of claim 1, wherein dynamically electroporating a population of cells located adjacent to the spatial region to transfer the treatment material into the population of cells comprises:
delivering, via the plurality of electroporation electrodes, electroporation signals to the population of cells to generate a dynamic electroporation electrical field proximate to the population of cells with the plurality of electroporation electrodes, wherein a location of a locus of the dynamic electroporation electrical field experiences a time-varying change through the spatial region.

3. The method of claim 2, further comprising:
while delivering the electroporation signals to the population of cells, moving the carrier member in a distal direction from a first position proximal to an external environment of the recipient to a second position distal to the external environment to create the time-varying change in the location of the locus of the dynamic electroporation electrical field through the spatial region.

4. The method of claim 2, further comprising:
while delivering the electroporation signals to the population of cells, moving the carrier member in a proximal direction from a second position distal to an external environment of the recipient to a first position proximal to the external environment to create the time-varying change in the location of the locus of the dynamic electroporation electrical field through the spatial region.

5. The method of claim 2, wherein delivering electroporation signals to the population of cells comprises:
delivering a series of biphasic current pulses to the population of cells.

6. The method of claim 1, wherein dynamically electroporating a population of cells located adjacent to the spatial region to transfer the treatment material into the population of cells comprises:
moving the carrier member along a path through the spatial region while simultaneously delivering a series of current pulses from an external electroporation device to the plurality of electroporation electrodes.

7. (canceled)

8. The method of claim 6, further comprising:
varying, at one or more locations in the spatial region, a rate at which the carrier member is moved through the spatial region.

9. The method of claim 8, wherein the spatial region is a scala tympani of a cochlea of a recipient, and wherein the method further comprises:
reducing the rate at which the carrier member is moved through the spatial region near a location of the cochlea corresponding to one or more specific acoustic frequency ranges.

10. The method of claim 6, wherein moving the carrier member along the path through the spatial region while simultaneously delivering a series of current pulses from the external electroporation device to the plurality of electroporation electrodes comprises:
moving the carrier member along the path in a stepwise manner; and
applying one or more current pulses at each of a plurality of discrete positions along the path.

11. The method of claim 6, wherein moving the carrier member along the path through the spatial region while simultaneously delivering a series of current pulses from the external electroporation device to the plurality of electroporation electrodes comprises:
moving the carrier member along the path in a continuous manner; and
periodically applying current pulses from the external electroporation device to the plurality of electroporation electrodes as the carrier member is moved along the path.

12. (canceled)

13. The method of claim 1, wherein the plurality of electroporation electrodes are disposed at a distal end of the carrier member.

14. The method of claim 1, further comprising:
removing the carrier member from the spatial region; and
inserting a stimulating assembly comprising a plurality of stimulation electrodes into the spatial region, wherein the plurality of stimulation electrodes are configured to deliver stimulation signals to the population of cells of the recipient.

15. The method of claim 1, inserting a carrier member comprising a plurality of electroporation electrodes into a spatial region within the body of a recipient comprises:
inserting the carrier member into a scala tympani of the recipient.

16. The method of claim 1, wherein introducing a treatment material into the spatial region comprises:
delivering a solution comprising the treatment material to the spatial region.

17. The method of claim 16, further comprising:
bringing one or more of the plurality of electroporation electrodes or one or more portions of the carrier member adjacent to one or more of the plurality of electroporation electrodes in contact with the cells located adjacent to the spatial region to substantially seal the solution comprising the treatment material within the spatial region for a period of time.

18. The method of claim 16, wherein the carrier member comprises an elongate cannula extending a length of the carrier member, and wherein delivering a solution comprising the treatment material to the spatial region comprises:
delivering the solution comprising the treatment material to the spatial region via the elongate cannula.

19. A method, comprising:
positioning one or more electroporation electrodes within a spatial region adjacent to cells in a body of a recipient, wherein the one or more electroporation electrodes are electrically connected to an electroporation device;
delivering a treatment material configured for transfer into the cells of the recipient into the spatial region; and
applying a series of current pulses from the electroporation device to the one or more electroporation electrodes while simultaneously moving the one or more electroporation electrodes along a path through the spatial region.

20. The method of claim 19, further comprising:
positioning the one or more electroporation electrodes at a first proximal position within the spatial region; and
moving the one or more electroporation electrodes in a distal direction from the first proximal position to a second distal position within the recipient to electroporate cells adjacent to the path.

21. The method of claim 19, further comprising:
positioning the one or more electroporation electrodes at a second distal position within the spatial region; and
moving the one or more electroporation electrodes in a proximal direction from the second distal position to a first proximal position within the recipient to electroporate cells adjacent to the path.

22. The method of claim 19, wherein the one or more electroporation electrodes comprise a plurality of electroporation electrodes.

23. The method of claim 22, wherein the plurality of electroporation electrodes are disposed at a distal end of a carrier member.

24. The method of claim 19, wherein applying a series of current pulses from the electroporation device to the one or more electroporation electrodes comprises:
applying a series of biphasic current pulses.

25. The method of claim 19, wherein applying a series of current pulses from the electroporation device to the one or more electroporation electrodes comprises:
generating the series of current pulses at the external electroporation device; and
providing the series of current pulses to the one or more electroporation electrodes.

26. The method of claim 19, further comprising:
removing the one or more electroporation electrodes from the spatial region; and
inserting a stimulating assembly comprising a plurality of stimulation electrodes into the spatial region, wherein the plurality of stimulation electrodes are configured to deliver stimulation signals to the cells of the recipient.

27. The method of claim 19, wherein moving the one or more electroporation electrodes along a path through the spatial region comprises:
moving the one or more electroporation electrodes along the path in a stepwise manner; and
applying one or more current pulses at each of a plurality of discrete positions along the path.

28. The method of claim 19, wherein moving the one or more electroporation electrodes along a path through the spatial region comprises:
moving the one or more electroporation electrodes along the path in a continuous manner; and
periodically applying current pulses from the electroporation device to the electroporation electrodes as the one or more electroporation electrodes are moved along the path.

29. The method of claim 19, wherein delivering a treatment material into the spatial region comprises:
delivering a solution comprising the treatment material to the spatial region.

30. The method of claim 29, wherein the one or more electroporation electrodes are embedded in a carrier member, and wherein the method further comprises:
bringing the one or more electroporation electrodes or one or more portions of the carrier member adjacent to the one or more electroporation electrodes in contact with the cells in the body of the recipient to substantially seal the solution comprising the treatment material within the spatial region.

31. The method of claim 29, wherein the one or more electroporation electrodes are disposed in a carrier member comprising an elongate cannula extending a length of the carrier member, and wherein delivering a solution comprising the treatment material to the spatial region comprises:
delivering the solution comprising the treatment material to the spatial region via the elongate cannula.

32-44. (canceled)
